(19) United States
(12) Patent Application Publication
(10) Pub. No.: US 2026/0118263 A1
(43) Pub. Date: Apr. 30, 2026

(54) Coal Moisture Content Measurement Method and System

(71) Applicant: TIANJIN RESEARCH INSTITUTE FOR WATER TRANSPORT ENGINEERING.M.O.T, Tianjin (CN)

(72) Inventors: Shitao PENG, Tianjin (CN); Xun YU, Tianjin (CN); Dengming ZHOU, Tianjin (CN); Guoqiang MA, Tianjin (CN); De LU, Tianjin (CN)

(73) Assignee: TIANJIN RESEARCH INSTITUTE FOR WATER TRANSPORT ENGINEERING.M.O.T, Tianjin (CN)

(21) Appl. No.: 19/160,302

(22) PCT Filed: Jun. 18, 2025
(86) PCT No.: PCT/CN2025/101676

§ 371 Date: Aug. 27, 2025

(30) Foreign Application Priority Data
Jun. 28, 2024 (CN) 202410853170.8

(51) Int. Cl. G01N 21/3554; G01N 21/359; G01N 33/24

(52) U.S. Cl. CPC G01N 21/3554; G01N 21/359; G01N 33/246

(57) ABSTRACT

The present invention relates to the technical field of coal moisture content detection, and discloses a coal moisture content measurement method and system. The method comprises: obtaining absorbance data and moisture content data of coal samples to acquire an absorbance interval and a moisture content interval of the coal samples, as well as the correspondence between the absorbance data and the moisture content data; determining segmentation thresholds for the moisture content interval, and dividing the moisture content interval into a first fitting interval, a second fitting interval, and a third fitting interval according to the segmentation thresholds; respectively fitting moisture content data and corresponding absorbance data in different fitting intervals to obtain a first fitting curve, a second fitting curve, and a third fitting curve; integrating the three fitting curves to obtain a complete coal absorbance-moisture content fitted curve; and measuring moisture content of coal to be tested.

### TECHNICAL FIELD

The present invention relates to the technical field of coal moisture content detection, and particularly relates to a coal moisture content measurement method and system.

### BACKGROUND TECHNOLOGY

Coal is one of the most abundant fuels stored on the entire earth and is an indispensable energy source in social life. Compared with railway and road transportation, coal transportation via marine routes has greater advantages in cost-effectiveness and capacity. As a key link in transshipment, coal bulk terminals serve as distribution hubs for coal, ensuring the stability of energy supply. However, coal bulk terminals face safety and environmental issues such as spontaneous combustion of coal and dust pollution. The most common method for addressing the safety and environmental problems at bulk terminals is water spraying operations. Since different coals have different critical spontaneous combustion moisture contents and dust-raising moisture contents, excessive spraying will reduce coal quality and affect coal characteristics, while insufficient spraying cannot effectively control dust pollution and prevent spontaneous combustion of coal. Therefore, online detection of coal moisture content is crucial.

Existing techniques include solutions for detecting coal moisture content by combining near-infrared spectroscopy technology with fitting algorithms. However, the existing techniques using a single algorithm cannot meet the requirements for coal moisture content determination under different conditions, have poor adaptability to different coal types and moisture content ranges, cannot achieve high-precision determination, and suffer from low detection efficiency.

Therefore, there is a pressing need for a coal moisture content measurement method and system, which can improve the precision and accuracy of fitting results, take into account fitting efficiency, and have strong adaptability to different coal types and moisture content ranges.

### SUMMARY OF INVENTION

To solve the above technical problems, the present invention provides a coal moisture content measurement method and system, which can improve the precision and accuracy of fitting results, take into account fitting efficiency, and have strong adaptability to different coal types and moisture content ranges.

The present invention provides a coal moisture content measurement method and system, comprising the following steps:
S1: Obtaining the absorbance data and the moisture content data of the coal samples; where the coal samples comprise coking coal, chemical coal, and thermal coal;
S2: Based on the absorbance data and the moisture content data of the coal samples, obtaining an absorbance interval and a moisture content interval of the coal samples, as well as the correspondence between the absorbance data and the moisture content data of the coal samples;
S3: Determining segmentation thresholds for the moisture content interval, and dividing the moisture content interval into a first fitting interval, a second fitting interval, and a third fitting interval according to the segmentation thresholds;
S4: Respectively fitting the moisture content data and corresponding absorbance data in the first fitting interval, the second fitting interval, and the third fitting interval to obtain a first fitting curve, a second fitting curve, and a third fitting curve;
S5: Integrating the first fitting curve, the second fitting curve, and the third fitting curve to obtain a complete coal absorbance-moisture content fitted curve;
S6: Measuring the moisture content of coal to be tested through the coal absorbance-moisture content fitted curve.

Further, in S3, determining the segmentation thresholds for the moisture content interval comprises:

Determining a first segmentation threshold and a second segmentation threshold for the moisture content intervals based on a critical moisture content interval of the coal samples; where the first segmentation threshold is 4.36%, and the second segmentation threshold is 10.46%.

Further, in S3, dividing the moisture content interval into the first fitting interval, the second fitting interval, and the third fitting interval according to the segmentation thresholds comprises:

Dividing the moisture content interval of the coal samples into the first fitting interval, the second fitting interval, and the third fitting interval according to the first segmentation threshold and the second segmentation threshold; wherein the first fitting interval is from 0.93% to 4.36%, the second fitting interval is from 4.36% to 10.46%, and the third fitting interval is from 10.46% to 21.53%.

Further, in S4, respectively fitting the moisture content data and corresponding absorbance data in the first fitting interval, the second fitting interval, and the third fitting interval to obtain the first fitting curve, the second fitting curve, and the third fitting curve comprises:
S41: Fitting the moisture content data and corresponding absorbance data in the first fitting interval using a support vector machine algorithm to obtain the first fitting curve;
S42: Fitting the moisture content data and corresponding absorbance data in the second fitting interval using a Bernstein polynomial fitting algorithm to obtain the second fitting curve;
S43: Fitting the moisture content data and corresponding absorbance data in the third fitting interval using a neural network algorithm to obtain the third fitting curve.

Further, in S42, fitting the moisture content data and corresponding absorbance data in the second fitting interval using a Bernstein polynomial fitting algorithm to obtain the second fitting curve comprises:
S421: Determining the moisture content data contained in the second fitting interval and the absorbance data corresponding to the moisture content data;
S422: Preprocessing the absorbance data and the moisture content data by removing data groups not conforming to the positive correlation relationship between the absorbance data and the moisture content data, according to the positive correlation relationship between the absorbance data and the moisture content data;
S423: Determining an order and coefficients of the Bernstein polynomial fitting algorithm based on the preprocessed absorbance data and moisture content data;
S424: Performing regression fitting on the Bernstein polynomial using a scalar function regression method according to the order and coefficients to obtain the second fitting curve.

Further, in S43, fitting the moisture content data and corresponding absorbance data in the third fitting interval using a neural network algorithm, wherein the neural network used in the neural network algorithm comprises: an input layer, a hidden layer, and an output layer;
the input layer comprises two neurons for inputting corresponding absorbance data and moisture content data;
the output layer comprises one neuron for outputting the third fitting curve of the absorbance data and moisture content data;
a number of layers of the hidden layer is 2; and
the neural network algorithm employs a normalization function premnmx.

Further, in S5, integrating the first fitting curve, the second fitting curve, and the third fitting curve to obtain the complete coal absorbance-moisture content fitted curve comprises:
integrating the first fitting curve, the second fitting curve, and the third fitting curve using a transition function to obtain the complete coal absorbance-moisture content fitted curve; wherein, the transition function is an exponential decay function.

The present invention further provides a coal moisture content measurement system for executing the coal moisture content measurement method described above, the system comprising:
a data acquisition module configured to: obtain the absorbance data and the moisture content data of the coal samples, where the coal samples comprise coking coal, chemical coal, and power coal; and obtain an absorbance interval and a moisture content interval of the coal samples, as well as the correspondence between the absorbance data and the moisture content data of the coal samples, based on the absorbance data and the moisture content data of the coal samples;
a moisture content interval segmentation module connected to the data acquisition module, configured to: determine segmentation thresholds for the moisture content interval, and divide the moisture content interval into a first fitting interval, a second fitting interval, and a third fitting interval according to the segmentation thresholds;
a fitting algorithm module connected to the moisture content interval segmentation module, configured to: respectively fit the moisture content data and corresponding absorbance data in the first fitting interval, the second fitting interval, and the third fitting interval to obtain the first fitting curve, the second fitting curve, and the third fitting curve;
a fitting curve integration module connected to the fitting algorithm module, configured to: integrate the first fitting curve, the second fitting curve, and the third fitting curve to obtain the complete coal absorbance-moisture content fitted curve;
a measurement module connected to the fitting curve integration module, configured to: measure the moisture content of coal to be tested through the coal absorbance-moisture content fitted curve.

Embodiments of the present invention have the following technical effects:

By segmenting the coal moisture content interval based on a critical moisture content interval of coal, then combining multiple fitting algorithms to fit coal absorbance and moisture content, high-precision fitting results for each interval segment are obtained; the fitting results of the three interval segments are integrated through a transition function to derive a final coal moisture content calculation model. This approach not only improves the precision and accuracy of fitting results but also takes into account fitting efficiency, exhibiting strong adaptability to different coal types and moisture content ranges.

### DESCRIPTION OF DRAWINGS

To more clearly illustrate the specific embodiments of the present invention or technical solutions in the prior art, the accompanying drawings required for describing the specific embodiments or the prior art will be briefly introduced below. Obviously, the drawings in the following description are some embodiments of the present invention. For those skilled in the art, other drawings may be obtained based on these drawings without creative efforts.

FIG. 1 is a flowchart of a coal moisture content measurement method provided in an embodiment of the present invention;

FIG. 2 is a flowchart of fitting moisture content data and corresponding absorbance data in a first fitting interval using a support vector machine algorithm provided in an embodiment of the present invention;

FIG. 3 is a flowchart of fitting moisture content data and corresponding absorbance data in a second fitting interval using a Bernstein polynomial fitting algorithm provided in an embodiment of the present invention;

FIG. 4 is a schematic structural diagram of a coal moisture content measurement system provided in an embodiment of the present invention.

### EMBODIMENTS

To make the objectives, technical solutions, and advantages of the present invention clearer, the technical solutions of the present invention will be described comprehensively and thoroughly below. It is evident that the described embodiments are merely part of the embodiments of the invention, not all embodiments. All other embodiments obtained by those of ordinary skill in the art based on the embodiments herein without creative efforts shall fall within the protective scope of the invention.

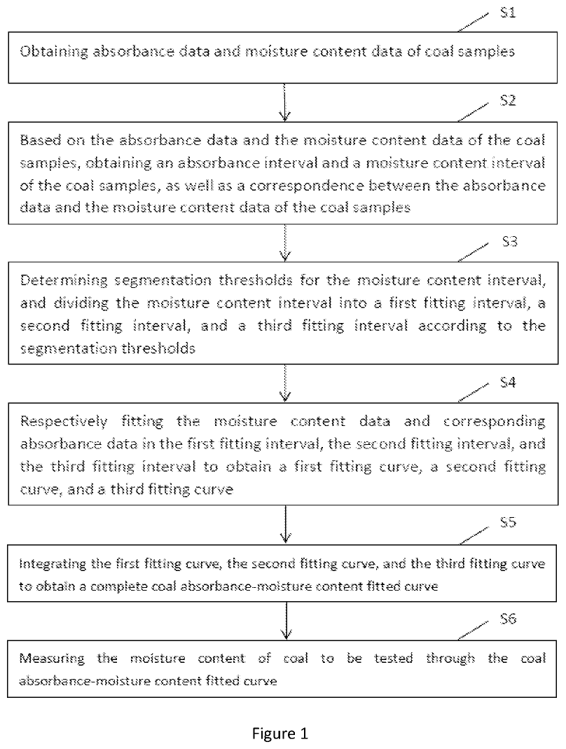

FIG. 1 is a flowchart of a coal moisture content measurement method according to an embodiment of the present invention. Referring to FIG. 1, the method specifically comprises:
S1: Obtain absorbance data and moisture content data of coal samples.

Specifically, the absorbance data of the coal samples is measured by a near-infrared method, and the moisture content data of the coal samples is measured by an international drying method. The coal samples include coking coal, chemical coal, and thermal coal.

Experimental procedures for measuring absorbance and moisture content using the near-infrared method and the national standard drying method are as follows:

instrumentation includes: a forced-air high-temperature drying oven; an analytical balance (weighing capacity 520 g, division value 0.1 mg); a desiccator, filter paper, and a near-infrared moisture analyzer.

Calibration steps: First weigh the desiccator to obtain weight A; next, place a 10 g coal sample on filter paper and measure its absorbance via the near-infrared method; transfer the coal sample from the filter paper into the desiccator, then weigh and record the combined weight as B on the analytical balance; place the desiccator in a drying oven at 105° C. for 3 hours; remove and cool for approximately 15 minutes, then reweigh and record the weight as B₁; return the desiccator to the drying oven for 30 minutes, remove and cool, then reweigh to obtain weight B₂; and repeat this cycle until the weight difference between two consecutive measurements is ≤1%.

The moisture content of the coal sample is calculated as follows:

$$y = \frac{C - A}{B - A} \times 100 \% ;$$

where A denotes the weight of the desiccator (g); B denotes the combined weight of the coal sample and the desiccator (g); C denotes the weight of the dried coal sample and the desiccator (g).

Further, preprocess the obtained absorbance data and moisture content data of the coal samples. The preprocessing includes data cleansing and normalization. The data cleansing is used to eliminate invalid or erroneous data; the normalization is used to eliminate deviations under different measurement conditions.
S2: Determine an absorbance interval and a moisture content interval of coal samples, and establish a correspondence between the absorbance data and the moisture content data based on the obtained data.

By way of example: the absorbance interval of coking coal is 0.860-0.963, corresponding to a moisture content interval of 0.93%-12.17%; the absorbance interval of chemical coal is 0.890-1.024, corresponding to a moisture content interval of 7.46%-21.53%; the absorbance interval of thermal coal is 0.878-1.001, corresponding to a moisture content interval of 4.52%-12.78%. Thus, the moisture content interval of the coal samples is determined to be 0.93%-21.53%. Based on this, establish a quantitative correlation between near-infrared signals and standard moisture content values within 0.93%-21.53%, i.e., the correspondence between the absorbance data and the moisture content data of the coal samples.
S3: Determine segmentation thresholds of the moisture content interval, and divide the moisture content interval into a first fitting interval, a second fitting interval, and a third fitting interval according to the thresholds.

Specifically, by compiling critical dust-suppression moisture content values: the critical moisture content intervals for coking coal, chemical coal, and thermal coal are 4.36%-5.00%, 10.00%-10.46%, and 8.00%-9.86% respectively and within these three intervals, coal properties remain unchanged. This indicates that maintaining coal moisture content at 4.36%-10.46% effectively suppresses spontaneous combustion and dust generation without affecting inherent properties. Determine a first segmentation threshold and a second segmentation threshold of the moisture content interval based on the critical moisture content intervals of the coal samples: the first segmentation threshold is 4.36%; and the second segmentation threshold is 10.46%.

Dividing the moisture content interval of the coal samples into the first fitting interval, the second fitting interval, and the third fitting interval according to the first segmentation threshold and the second segmentation threshold; where the first fitting interval is 0.93%-4.36%, the second fitting interval is 4.36%-10.46%, and the third fitting interval is 10.46%-21.53%.
S4: Perform fitting on moisture content data and corresponding absorbance data in the first fitting interval, the second fitting interval, and the third fitting interval respectively to obtain a first fitting curve, a second fitting curve, and a third fitting curve.
S41: Fit the moisture content data and corresponding absorbance data of the coal samples in the first fitting interval using the support vector machine algorithm to obtain the first fitting curve.

Specifically, the support vector machine algorithm is insensitive to outliers and noise. When the coal moisture content is low, dust generation readily occurs, which may adversely affect detection. Adopting the support vector machine algorithm reduces the impact of outliers and noise on fitting accuracy. Moreover, the support vector machine algorithm achieves effective learning with limited data. Even in the low moisture content interval with sparse data and small variations, it provides accurate predictions. Therefore, the support vector machine algorithm is adopted for fitting the first fitting interval.

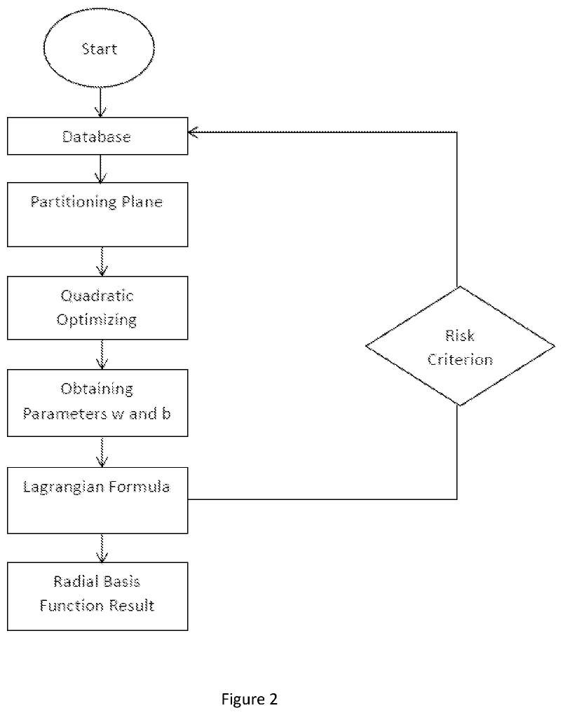

Specifically, FIG. 2 is a flowchart of fitting the moisture content data and the corresponding absorbance data in the first fitting interval using the support vector machine algorithm provided in an embodiment of the present invention. Referring to FIG. 2, when using the SVM algorithm, it is necessary to consider the risks of the Empirical Risk Minimization (ERM) criterion and the Structural Risk Minimization (SRM) criterion. The calculation formulas are as follows:

$${R}_{ERM} = \frac{1}{L} {\sum }_{i = 1}^{L} L ( {y}_{i} , f ( {x}_{i} ) ) ;$$

$${R}_{SRM} \leq {R}_{ERM} + \sqrt{\frac{h ( ln ( \frac{2 L}{h} + 1 ) - ln ( \frac{μ}{4} ) )}{L}} ;$$

where L represents the confidence range, h represents the interval difference, μ represents the error, xᵢ represents the i-th group of absorbance data, yᵢ represents the i-th group of moisture data, R_{ERM} represents the risk value of empirical risk minimization, and R_{SRM} represents the risk value of structural risk minimization. According to different linear problems, to ensure the accuracy of the target sample, radial organic function classification is used to detect the moisture content of coal. The calculation formula is as follows:

$$K \{ {x}_{\text{?}} , {y}_{\text{?}} ) = {R}_{SRM} \vert {[ ( {x}_{\text{?}} , {y}_{\text{?}} ) + 1 ]}^{\text{?}} \vert ;$$

$$\text{?} \text{indicates text missing or illegible when filed}$$

where K represents the inner product of the spatial vectors, and n represents the order of the polynomial.

The present solution uses a linear function to partition the moisture content data and corresponding absorbance data of the coal samples in the first fitting interval, thus dividing the data points. Support vector machine parameters are selected, and quadratic optimization conditions are formulated and solved to obtain the correlation coefficient. Finally, fitting is performed to obtain the final result. The formula for establishing the partitioning plane is as follows:

$$w \times {x}_{i} + b = 0 ;$$

where w is the normal vector, which determines the direction of the hyperplane; b represents the displacement.

Partitioning the database requires the following:

$${y}_{\text{?}} ( {wx}_{\text{?}} - b ) \geq 1 , i = 1 , … n ;$$

$$\text{?} \text{indicates text missing or illegible when filed}$$

the partitioning result is obtained by selecting the parameter w²/2, and the given conditions are:

$${y}_{\text{?}} ( {wx}_{\text{?}} + b ) - 1 \geq 0 , i = 1 , 2 , … , n ;$$

$$\text{?} \text{indicates text missing or illegible when filed}$$

under given conditions, the vector of parameter w²/2 is w, and it is calculated using the Lagrange formula, which is as follows:

$$L = \frac{1}{2} {w}^{2} - {R}_{SRM} {\sum }_{i = 1}^{n} [ {y}_{n} ( {wx}_{\text{?}} + b ) - 1 ] ;$$

$$\text{?} \text{indicates text missing or illegible when filed}$$

and
the optimal radial organic function is derived through the above formula, thereby obtaining the first fitting curve.
S42: Use a Bernstein polynomial fitting algorithm to fit the moisture content data of coal samples and their corresponding absorbance data in the second fitting interval, resulting in the second fitting curve.

Specifically, the Bernstein polynomial fitting algorithm allows for precise control over the fitting curve. The moisture content interval is the critical range for the coal moisture content, and it is necessary to accurately describe the relationship between moisture content and absorbance, with high accuracy requirements. Therefore, the Bernstein polynomial fitting algorithm is used for fitting in the second fitting interval.

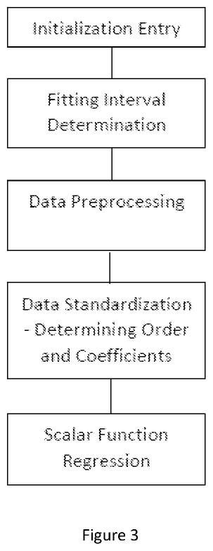

Specifically, FIG. 3 is a flowchart of fitting the moisture content data and the corresponding absorbance data in the second fitting interval using the Bernstein polynomial fitting algorithm provided in an embodiment of the present invention. Referring to FIG. 3:
S421: Determine the moisture content data included in the second fitting interval and the corresponding absorbance data.
S422: Based on the positive correlation relationship between the absorbance data and the moisture content data, preprocessing the absorbance and moisture content data by removing data groups that do not follow the positive correlation relationship.

For example, since the absorbance data and moisture content data are positively correlated, that is, when the absorbance data x₁ is smaller than the absorbance data x₂, the corresponding moisture content data y₁ should also be smaller than the moisture content data y₂. Assuming the absorbance data relationship is x₁<x₂<x₃, and the corresponding moisture content data relationship is y₂<y₁<y₃, then the absorbance-moisture content data set x₂-y₂ does not conform to the positive correlation relationship and needs to be eliminated. Similarly, all absorbance-moisture content data that do not conform to the positive correlation relationship are eliminated, ensuring that the remaining absorbance-moisture content data all conform to the positive correlation relationship:

x₁<x₂<x₃ . . . <xₙ→y₁<y₂<y₃ . . . <yₙ, thereby filtering out outliers and improving fitting accuracy.
S423: Determine the order and coefficients of the Bernstein polynomial fitting algorithm based on the preprocessed absorbance and moisture content data.

Specifically, the Bernstein polynomial is preliminarily determined based on the pre-processed absorbance data and moisture content data. The formula is as follows:

$${B}_{m} ( {x}_{1} , … , {x}_{n} \vert A \} = {\sum }_{{{k}_{1} = 0}^{}}^{m} … {\sum }_{{k}_{n} = 0}^{m} {A}_{{k}_{1} {k}_{2} … {k}_{n}} {p}_{{mk}_{1}} ( {x}_{1} ) … {p}_{{mk}_{n}} ( {x}_{n} ) ;$$

Where m represents the order, x₁, . . . , xₙ represent the absorbance data, n represents the total number of data points, A represents the undetermined coefficient, A=(Aₖ_{₁}_{,k}_{₂}_{. . . k}_{ₙ}, i=1, 2, . . . n,kₙ=0, 1, . . . , m); and Pₘ represents a set of no more than m algebraic polynomials.

Based on the definition of an m-order Bernstein polynomial and simple algebra, the definition of a recursive polynomial can be written as:

$$( 1 - x ) {B}_{n , m - 1} ( x ) + … + {xB}_{n - 1 , m - 1} ( x ) = ( 1 - x ) ( \begin{matrix}m - 1 \\ n\end{matrix} ) {{x}^{n} ( 1 - x )}^{m - 1 - n} + … + x ( \begin{matrix}m - 1 \\ n - 1\end{matrix} ) {{x}^{n - 1} ( 1 - x )}^{m - 1 - ( n - 1 )} = ( \begin{matrix}m - 1 \\ n\end{matrix} ) {{x}^{n} ( 1 - x )}^{m - n} + … + ( \begin{matrix}m - 1 \\ n - 1\end{matrix} ) {{x}^{n} ( 1 - x )}^{m - n} = [ ( \begin{matrix}m - 1 \\ n\end{matrix} ) + … + ( \begin{matrix}m - 1 \\ n - 1\end{matrix} ) ] {{x}^{n} ( 1 - x )}^{m - n} = ( \begin{matrix}m \\ n\end{matrix} ) {{x}^{n} ( 1 - x )}^{m - n} ;$$

where, B_{n,m-1} represents the nth m-th Bernstein polynomial obtained by mixing multiple m−1-order Bernstein polynomials. This formula is used to demonstrate the recursive nature of Bernstein polynomials.

Using the power basis exponent to determine the m-th order, and using the binomial as an example, we obtain the following formula:

$${B}_{n , m} ( x ) = ( \begin{matrix}m \\ n\end{matrix} ) {{x}^{n} ( 1 - x )}^{m - n} = ( \begin{matrix}m \\ n\end{matrix} ) {x}^{n} {\sum }_{i = 1}^{m - n} {( - 1 )}^{i} ( \begin{matrix}m - n \\ i\end{matrix} ) {x}^{i} = {\sum }_{i = 0}^{m - n} {( - 1 )}^{i} ( \begin{matrix}m \\ n\end{matrix} ) ( \begin{matrix}m - n \\ i\end{matrix} ) {x}^{i + n} = {\sum }_{i = n}^{m} {( - 1 )}^{i - n} ( \begin{matrix}m \\ n\end{matrix} ) ( \begin{matrix}m - n \\ i - n\end{matrix} ) {x}^{i} = {\sum }_{i = n}^{m} {( - 1 )}^{i - n} ( \begin{matrix}m \\ i\end{matrix} ) ( \begin{matrix}i \\ n\end{matrix} ) {x}^{i} ;$$

where B_{n,m} represents the n-th Bernstein polynomial of order m, and this formula can be derived from the definition of the Bernstein polynomial or the binomial theorem.

The power basis [1, x, x², . . . , xⁿ] forms the basis of the polynomial space of degree less than or equal to m. Therefore, any m-th order Bernstein polynomial can be expressed using the power basis. By converting the power basis to the Bernstein basis, the order m is determined using the upward-order formula, as shown below:

$${x}^{n} = x ( {x}^{n - 1} ) = x {\sum }_{i = n - 1}^{m} \frac{( \begin{matrix}i \\ n - 1\end{matrix} )}{( \begin{matrix}m \\ n - 1\end{matrix} )} {B}_{i , m - 1} ( x ) = {\sum }_{i = n}^{m} \frac{( \begin{matrix}i - 1 \\ n - 1\end{matrix} )}{( \begin{matrix}m - 1 \\ n - 1\end{matrix} )} {xB}_{i - 1 , m - 1} ( x ) = {\sum }_{i = n - 1}^{m - 1} \frac{( \begin{matrix}i \\ n - 1\end{matrix} )}{( \begin{matrix}m \\ n - 1\end{matrix} )} \frac{i}{n} {B}_{i , m} ( x ) = {\sum }_{i = n - 1}^{m - 1} \frac{( \begin{matrix}i \\ n\end{matrix} )}{( \begin{matrix}m \\ n\end{matrix} )} {B}_{i , m} ( x ) ;$$

where B_{i, m} represents the number of the i-th m-th order Bernstein polynomial.

Based on the matrix form of the absorbance-water content data points, the polynomial coefficients of the linear combination of the Bernstein basis functions are determined using the following formula:

$$B ( x ) = [ {B}_{0 , m} ( x ) , {B}_{1 , m} ( x ) , … , {B}_{n , m} ( x ) ] [ \begin{matrix}{k}_{0} \\ {k}_{1} \\ ⋮ \\ {k}_{n}\end{matrix} ] ;$$

This formula is an m-order matrix representation, expressed as a vector dot product. B(x) represents the polynomial, and B_{n, m}(x) represents the m-order data used to determine the power basis coefficients of the corresponding Bernstein polynomial.

Based on the power basis, the above formula can be written as:

$$B ( x ) = [ 1 , x , {x}^{2} , … {x}^{m} ] [ \begin{matrix}{y}_{0 , 0} & 0 & 0 & … & 0 \\ {y}_{1 , 0} & {y}_{1 , 1} & 0 & … & 0 \\ ⋮ & ⋮ & ⋮ & ⋱ & ⋮ \\ {y}_{n , 0} & {y}_{n , 1} & {y}_{n , 2} & … & {y}_{n , m}\end{matrix} ] [ \begin{matrix}{k}_{0} \\ {k}_{1} \\ ⋮ \\ {k}_{n}\end{matrix} ] ;$$

S424: Based on the order and coefficients, use a scalar function regression to perform regression fitting on the Bernstein polynomial to obtain the second fitting curve.

Specifically, a scalar function regression is used to perform regression fitting on the Bernstein polynomial, combining the coefficients and order calculated using the above formula to ultimately obtain the second fitting curve for the coal samples.

$${Y}_{i} = α + \int {B}_{m} ( x ) dx = {\sum }_{i = 1}^{n} {\sum }_{{k}_{n} = 0}^{m} {A}_{{K}_{n}} {P}_{{mk}_{n}} ( {x}_{i} ) + α$$

where Yᵢ represents the second fitting curve obtained ultimately, a represents a scalar constant, and m represents the order.
S43: For the third fitting interval, use a neural network algorithms to fit the moisture content data of the coal samples and the corresponding absorbance data to obtain the third fitting curve.

Specifically, neural network algorithms excel at capturing and learning nonlinear relationships between input data (e.g., absorbance) and output data (e.g., moisture content), and are well suited to handling the significant nonlinear relationships in high moisture content intervals. Furthermore, neural network algorithms are computationally simple, and for high moisture content intervals with a large amount of data, they can maintain both accuracy and efficiency. Therefore, the neural network algorithm is used to fit the third fitting interval.

Specifically, the neural network adopted by the neural network algorithm comprises: an input layer, a hidden layer, and an output layer;
the input layer includes two neurons for inputting corresponding absorbance data and moisture content data;
the output layer includes one neuron for outputting the third fitting curve of the absorbance data and moisture content data;
a number of layers of the hidden layer is 2; and
the neural network algorithm employs the normalization function premnmx.

For a neuron processing unit, the input value of is uᵢ. When it passes through the hidden layer, it compares and filters the data. The output value U is combined with the strength weight Wᵢ of the interaction between processing units and the internal threshold q.

The input of the neuron is:

$${\sum }_{i = 1}^{n - 1} {W}_{i} , {u}_{i} ;$$

the output of the neuron is:

$$U = f ( {\sum }_{i = 1}^{n - 1} {W}_{i} , {u}_{i} - q ) ;$$

where f is an action function.
S5. Integrate the first fitting curve, the second fitting curve, and the third fitting curve to obtain a complete coal absorbance-moisture content fitting curve.

Specifically, the first, second, and third fitting curves are integrated using a transition function to obtain the complete coal absorbance-moisture content fitting curve; the transition function is an exponential decay function.

The calculation formula for integrating the fitting curves of two adjacent fitting intervals using the exponential decay function is as follows:

$$f ( x ) = a + ( b - a ) \times {e}^{- λ \times ( x - c )} ;$$

where λ represents the decay rate, a is the end point of the first fitting curve, b is the initial end point of the second fitting curve, c is the center point where the function transitions from a to b, and x represents the independent variable parameter.

The three fitting curves are integrated according to the above formula to obtain a complete coal absorbance-moisture content fitting curve.
S6: Measure the moisture content of the coal to be tested using the coal absorbance-moisture content fitting curve.

The present embodiment of the present invention divides the coal moisture content range into segments based on the critical range of coal moisture content. Then, multiple fitting algorithms are used to fit the coal absorbance and moisture content, resulting in high-precision fitting results for each segment. The fitting results for the three segments are integrated using a transition function to obtain the final coal moisture content calculation model. This not only improves the precision and accuracy of the fitting results, but also takes into account fitting efficiency, and has strong adaptability to different coal types and moisture content ranges.

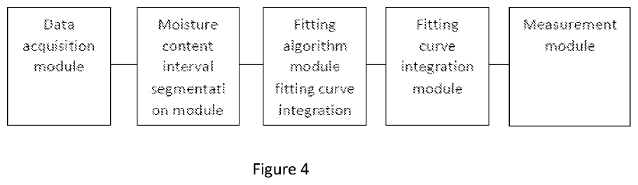

FIG. 4 is a schematic structural diagram of a coal moisture content measurement system provided in an embodiment of the present invention. This system is used to implement the coal moisture content measurement method described in the above embodiment. As shown in FIG. 4, the system includes the following modules:
a data acquisition module configured to: obtain the absorbance data and the moisture content data of the coal samples, where the coal samples comprise coking coal, chemical coal, and power coal; and obtain an absorbance interval and a moisture content interval of the coal samples, as well as the correspondence between the absorbance data and the moisture content data of the coal samples, based on the absorbance data and the moisture content data of the coal samples;
a moisture content interval segmentation module connected to the data acquisition module, configured to: determine segmentation thresholds for the moisture content interval, and divide the moisture content interval into a first fitting interval, a second fitting interval, and a third fitting interval according to the segmentation thresholds;
a fitting algorithm module connected to the moisture content interval segmentation module, configured to: respectively fit the moisture content data and corresponding absorbance data in the first fitting interval, the second fitting interval, and the third fitting interval to obtain the first fitting curve, the second fitting curve, and the third fitting curve;
a fitting curve integration module connected to the fitting algorithm module, configured to: integrate the first fitting curve, the second fitting curve, and the third fitting curve to obtain the complete coal absorbance-moisture content fitted curve;
a measurement module connected to the fitting curve integration module, configured to: measure the moisture content of coal to be tested through the coal absorbance-moisture content fitted curve.

It should be noted that the terms used in the present invention are only for describing specific embodiments and are not intended to limit the scope of the present application. As shown in the description of the present invention, unless the context explicitly suggests otherwise, terms such as “one,” “a,” “an,” and/or “the” do not refer to singular forms specifically and may also include the plural. The terms “comprise,” “include,” or any of their variations are intended to encompass a non-exclusive inclusion, so that a process, method, or device including a series of elements not only comprises those elements but also includes other elements not explicitly listed or inherent to such a process, method, or device. In the absence of further limitations, elements defined by the phrase “comprising one . . . ” do not exclude the presence of additional identical elements in a process, method, or device that includes the said elements.

It should also be clarified that terms such as “center,” “up,” “down,” “left,” “right,” “vertical,” “horizontal,” “internal,” “external,” and other directional or positional relations refer to the positions or orientations shown in the drawings and are used for the convenience of describing the present invention and simplifying the description. These terms do not indicate or imply that the device or component referred to must have a specific orientation or be constructed and operated in a specific direction, and therefore should not be understood as limiting the present invention. Unless otherwise explicitly defined, terms such as “mounted,” “connected,” “coupled,” etc., should be broadly understood. For example, connections can be fixed, detachable, or integrally connected; they can be mechanical or electrical connections; they can be direct connections or connected through an intermediate medium; they can also refer to internal communication between two elements. Those skilled in the art can interpret the specific meaning of these terms in the context of the present invention.

Finally, it should be noted that the above embodiments are only for illustrating the technical solutions of the present invention and are not intended to limit the present invention. Although the present invention has been described in detail with reference to the aforementioned embodiments, one of ordinary skill in the art should understand that modifications can be made to the technical solutions described in these embodiments, or some or all of the technical features can be equivalently replaced. Such modifications or replacements do not depart from the essence of the present invention's technical solutions.

1. A coal moisture content measurement method, comprising following steps:
S1: Obtaining absorbance data and moisture content data of coal samples; wherein the coal samples comprise coking coal, chemical coal, and thermal coal;
S2: Based on the absorbance data and the moisture content data of the coal samples, obtaining an absorbance interval and a moisture content interval of the coal samples, as well as a correspondence between the absorbance data and the moisture content data of the coal samples;
S3: Determining segmentation thresholds for the moisture content interval, and dividing the moisture content interval into a first fitting interval, a second fitting interval, and a third fitting interval according to the segmentation thresholds;
S4: Respectively fitting the moisture content data and corresponding absorbance data in the first fitting interval, the second fitting interval, and the third fitting interval to obtain a first fitting curve, a second fitting curve, and a third fitting curve specifically comprises:
S41: Fitting the moisture content data and corresponding absorbance data in the first fitting interval using a support vector machine algorithm to obtain the first fitting curve;
S42: Fitting the moisture content data and corresponding absorbance data in the second fitting interval using a Bernstein polynomial fitting algorithm to obtain the second fitting curve; and
S43: Fitting the moisture content data and corresponding absorbance data in the third fitting interval using a neural network algorithm to obtain the third fitting curve;
S5: Integrating the first fitting curve, the second fitting curve, and the third fitting curve to obtain a complete coal absorbance-moisture content fitted curve specifically comprises:
integrating the first fitting curve, second fitting curve, and third fitting curve through a transition function to obtain the complete coal absorbance-moisture content fitting curve; wherein the transition function is an exponential decay function; and
S6: Measuring the moisture content of coal to be tested through the coal absorbance-moisture content fitted curve.

2. The coal moisture content measurement method according to claim 1, wherein in S3, determining the segmentation thresholds for the moisture content interval comprises:
determining a first segmentation threshold and a second segmentation threshold for the moisture content intervals based on a critical moisture content interval of the coal samples; wherein the first segmentation threshold is 4.36%, and the second segmentation threshold is 10.46%.

3. The coal moisture content measurement method according to claim 2, wherein in S3, dividing the moisture content interval into the first fitting interval, the second fitting interval, and the third fitting interval according to the segmentation thresholds comprises:
dividing the moisture content interval of the coal samples into the first fitting interval, the second fitting interval, and the third fitting interval according to the first segmentation threshold and the second segmentation threshold; wherein the first fitting interval is from 0.93% to 4.36%, the second fitting interval is from 4.36% to 10.46%, and the third fitting interval is from 10.46% to 21.53%.

4. The coal moisture content measurement method according to claim 1, wherein in S42, fitting the moisture content data and corresponding absorbance data in the second fitting interval using a Bernstein polynomial fitting algorithm to obtain the second fitting curve comprises:
S421: Determining the moisture content data contained in the second fitting interval and the absorbance data corresponding to the moisture content data;
S422: Preprocessing the absorbance data and the moisture content data by removing data groups not conforming to the positive correlation relationship between the absorbance data and the moisture content data, according to the positive correlation relationship between the absorbance data and the moisture content data;
S423: Determining an order and coefficients of the Bernstein polynomial fitting algorithm based on the preprocessed absorbance data and moisture content data; and
S424: Performing regression fitting on the Bernstein polynomial using a scalar function regression method according to the order and coefficients to obtain the second fitting curve.

5. The coal moisture content measurement method according to claim 4, wherein in S43, fitting the moisture content data and corresponding absorbance data in the third fitting interval using a neural network algorithm, wherein the neural network used in the neural network algorithm comprises: an input layer, a hidden layer, and an output layer;
the input layer comprises two neurons for inputting corresponding absorbance data and moisture content data;
the output layer comprises one neuron for outputting the third fitting curve of the absorbance data and moisture content data;
a number of layers of the hidden layer is 2; and
the neural network algorithm employs a normalization function premnmx.

6. A coal moisture content measurement system for executing the coal moisture content measurement method according to claim 1, wherein the system comprises:
a data acquisition module, used to obtain the absorbance data and the moisture content data of the coal samples, wherein the coal samples comprise coking coal, chemical coal, and power coal; and obtain an absorbance interval and a moisture content interval of the coal samples, as well as the correspondence between the absorbance data and the moisture content data of the coal samples, based on the absorbance data and the moisture content data of the coal samples;
a moisture content interval segmentation module connected to the data acquisition module, used to determine segmentation thresholds for the moisture content interval, and divide the moisture content interval into a first fitting interval, a second fitting interval, and a third fitting interval according to the segmentation thresholds;
a fitting algorithm module connected to the moisture content interval segmentation module, used to respectively fit the moisture content data and corresponding absorbance data in the first fitting interval, the second fitting interval, and the third fitting interval to obtain the first fitting curve, the second fitting curve, and the third fitting curve; specifically comprising: fitting the moisture content data and corresponding absorbance data in the first fitting interval using a support vector machine algorithm to obtain the first fitting curve; fitting the moisture content data and corresponding absorbance data in the second fitting interval using a Bernstein polynomial fitting algorithm to obtain the second fitting curve; and fitting the moisture content data and corresponding absorbance data in the third fitting interval using a neural network algorithm to obtain the third fitting curve;
a fitting curve integration module connected to the fitting algorithm module, used to integrate the first fitting curve, the second fitting curve, and the third fitting curve to obtain the complete coal absorbance-moisture content fitted curve; specifically comprising: integrating the first fitting curve, second fitting curve, and third fitting curve through a transition function to obtain the complete coal absorbance-moisture content fitting curve; wherein the transition function is an exponential decay function; and
a measurement module connected to the fitting curve integration module, used to measure the moisture content of coal to be tested through the coal absorbance-moisture content fitted curve.
